(19) United States
(12) Patent Application Publication
(10) Pub. No.: US 2012/0123199 A1
(43) Pub. Date: May 17, 2012

(54) DEVICE FOR SEXUAL STIMULATION WITH SUPPLEMENTARY ELEMENT (VARIANTS)

(76) Inventor: Sergei E. Topolev, St.-Petersburg (RU)

(21) Appl. No.: 13/124,293

(22) PCT Filed: Oct. 12, 2009
(86) PCT No.: PCT/RU09/00553

§ 371 Date: Sep. 19, 2011

(30) Foreign Application Priority Data
Oct. 14, 2008 (RU) 2008141319

(51) Int. Cl. A61F 5/00

(52) U.S. Cl. 600/38

(57) ABSTRACT

The device for stimulating erogenous zones comprises a dildo and at least one additional stimulating element (ASE) attached to the dildo and including a body of rotation around an axis that is close to normal relative to a longitudinal axis of the dildo. A surface of the ASE projects above the dildo outer surface so that the ASE is able to roll over a surface of the erogenous zones. The ASE may include a ball or a cylinder. The surface of the ASE can be made relief. The device can be provided with a plurality of the ASE and at least a portion of the dildo can include a spiral frame, with the spiral fed through the plurality of the ASE.

The present invention relates to medical engineering and the section of Sexology in particular. It relates to personal use devices that enhance sexual desire and facilitate the achievement of women's orgasm, and can be used for correction of sexual disharmony.

The analogue of the invention is a vibrator with additional clitoral stimulator known as “rabbit.” Its disadvantage lies in its inability to perform reciprocating motions in a considerable depth inside of the vagina with simultaneous continuous stimulation of the clitoris, as the clitoral stimulator is attached to the rear side of the vibrator.

The essential features of the invention are:

Additional element that moves along the longitudinal axis of the dildo, vibrator or similar device (the “dildo”) and contains an element for clitoral stimulation, which may include a vibrator or vacuum stimulator or round shape stimulator placed on the axis of the additional element close to the perpendicular of the longitudinal axis of the dildo, and with its one side pressed against the dildo and the side that is close to a right angle from the mentioned side facing the clitoris; and the device for pressing the element for clitoral stimulation against external genitalia, therefore the additional element is spring-loaded to the rear side of the dildo with a spring or corrugated tube or contains a haul on the front of the dildo or has an arrangement for fixing the device on the body or holding it with hands. A wavelike pilot hole or a ledge placed along the axis on the body of the dildo for transmission of the mechanical motion to the additional element with a clitoral stimulator or to the clitoral stimulator when the additional element moves along the dildo, which is enabled by the additional element or clitoral element containing a holdfast fixing it into the wavelike pilot holes or ledges. Notably, the dildo or vibrator may contain multiple wavelike grooves or ledges that enable different kinds of movements of the clitoral element, and have replaceable guide paths or a fixing mechanism allowing to choose between the various guides.

The technical result would be enhanced sexual sensations for women and facilitation in achieving of an orgasm when the dildo or vibrator moves in a considerable depth inside the vagina, due to the additional continuous clitoral stimulation which is achieved through constant contact of the clitoral element with external genitalia, as well as through vibrational or vacuum clitoral stimulation, and through reciprocating straight, angular or circular motions of the clitoral element that moves up and down, left or right relatively to the genital slit, or through other movements, such as tapping.

Additional element placed on the dildo or vibrator with the rotation axis close to the perpendicular of the longitudinal axis of the dildo with its outer surface protruding above the body of the dildo that may have relief outer surface.

Additional element with eccentric axis of rotation.

Elongated additional element which outer surfaces face each other at a wide angle close to 150 degrees with the axis of rotation close to the center, which can be spring loaded or pressed at one end with an elastic element.

Several additional elements (all same or a combination of different ones) placed on the dildo.

Body frame of the dildo or a part of it with several additional elements mounted on it and rotating around their axis along the axis close to the perpendicular of the longitudinal axis of the dildo, in particular, shaped like a spiral.

The technical result would be enhanced female sexual sensations and facilitation in achieving an orgasm when the moving dildo is placed inside of the vagina: due to the additional stimulation caused by the rotating element with embossed surface; due to the additional stimulation caused by the additional element with an eccentric axis which creates ‘tapping effect’; due to reduced friction in the presence of the additional element with a large outer surface or a few additional rolling elements that enable the dildo to move with a ‘rolling effect’ when there is insufficient lubrication or during stimulation of the external genitalia through clothing; due to enhanced stimulation of the G-point by the elongated additional element front end of which is lifted over the dildo and pressed against the G-point when the dildo is inserted into the vagina, and the rear end of the additional element is pressed against the dildo because of the narrowed vaginal orifice: vaginal muscles training by using one end of the elongated element which can be spring-loaded or forcibly pressed against with its elastic element or by hand.

Additional element for stimulation of internal or external or internal and external erotogenic zones of female genitalia, which consists of the stimulating part and the attachment part and mounted on the dildo with a locking mechanism that consists of a pass-through or blind hole or recess on the dildo or vibrator along the axis close to the perpendicular of the longitudinal axis of the dildo and a through slit from the hole or recess to the outer surface of the dildo or vibrator. Notably, the width of the slit is smaller than the diameter or width of the hole or recess, and the attachment part of the additional element is designed as a counterpart of the locking mechanism of the dildo or vibrator, so that the counterpart of the additional element is inserted into the locking mechanism of the dildo by moving the counterpart of the additional element along the axis of the hole or recess with a through slit and is kept there due to the slits that are smaller than the hole or recess, whereas the end of the attachment part of the additional element is thickened.

The invention is aimed at satisfying woman's sexual needs. It allows women who are unable to experience vaginal orgasm to achieve sexual gratification through clitoral orgasm during simultaneous vaginal stimulation; experience extra sexual sensations through vaginal stimulation performed by the additional axial elements; increase their sexual arousal by vaginal penetration without friction in case of insufficient lubrication; achieve an orgasm through stimulation of the external genitalia through clothing; experience additional stimulation of the G-point; train vaginal muscles; use additional removable elements for a variety of sexual sensations.

The invention consists of a vaginal element that has the size and shape similar to those of an erected penis, which makes it suitable for its insertion into the vagina and for vaginal stimulation, and that may include vibrating mechanism; an additional element that can move freely along the vaginal element with the clitoral stimulator and has the size and shape suitable for stimulation of external genitalia; pressing down unit which provides constant contact between the clitoral stimulator and external genitalia while the vaginal element is moving inside the vagina. Vaginal element and the additional axial element that protrudes partially or completely above the surface of the vaginal element or several vaginal elements with full or limited rotation around their axis. Vaginal element with the additional removable element or elements for stimulation of external and/or internal genitalia.

The device is made of material or materials that meet the product design features, such as plastic, silicone, metal, glass, natural materials, etc.

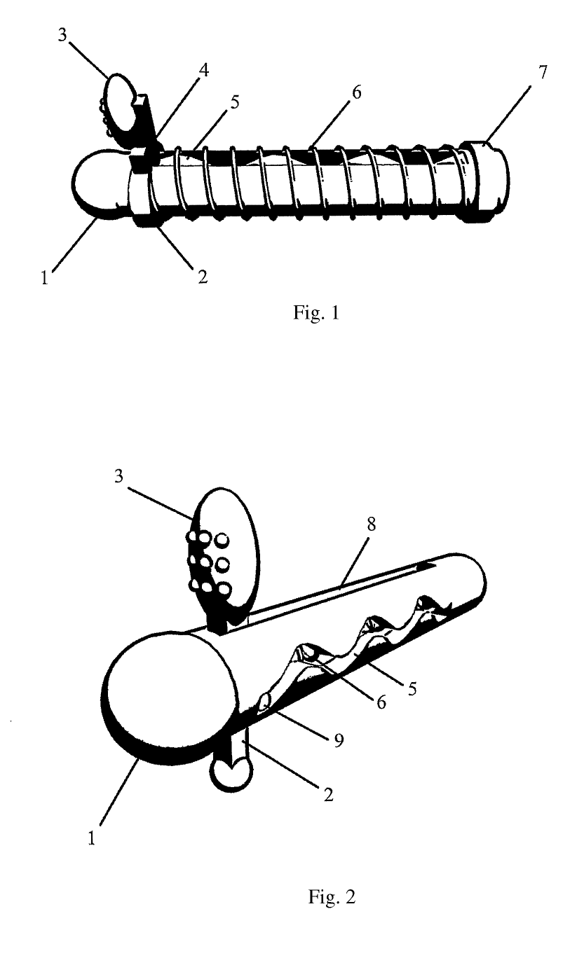

FIG. 1 shows the version of the invention with the reciprocating clitoral element that moves along the axis close to perpendicular of the longitudinal axis of the dildo or vibrator (the “dildo”), imitating the manual stimulation along the genital slit in up-and-down motion when it moves along the dildo, therefore the dildo contains wavelike longitudinal recess, body frame of the additional element with the attached clitoral element. Notably, the clitoral element moves along the axis close to the perpendicular of the longitudinal axis of the dildo and it is spring-loaded to the wavelike groove, and the pressing down unit, which is an outer spring from the rear end of the dildo, provides contact between clitoral element and external genitalia when the dildo moves inside the vagina.

FIG. 2 shows the version of the invention with the reciprocating clitoral element that moves along the axis close to perpendicular of the longitudinal axis of the dildo or vibrator, imitating manual stimulation along the genital slit in up-and-down motion when it moves along the dildo, therefore the dildo contains through symmetrical wavelike slits and a straight longitudinal slit in which the body frame of the additional element with the clitoral element is placed, and the pressing down unit, which is an inner spring from the rear end of the dildo, provides contact between clitoral element and external genitalia when the dildo moves inside the vagina.

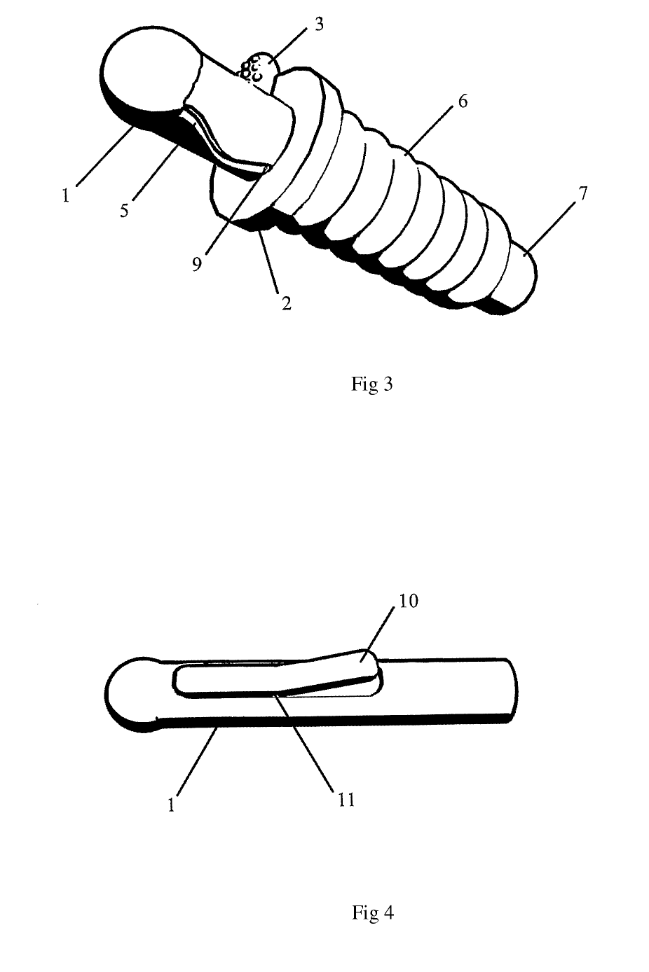

FIG. 3 shows the version of the invention with the clitoral element moving in a reciprocating angular motion relatively to the longitudinal axis of the dildo, which imitates manual stimulation performed across the genital slit in a right-left motion as it moves along the dildo, therefore the dildo has a wavelike groove that is shaped like a small canal with undulating profile, and the body frame of the additional element placed on the dildo is shaped like a ring with a ledge on its inner side which serves as a locking mechanism, and the pressing mechanism placed on the dildo is shaped like a corrugated tube with one end pressed against the body of the additional element and the other end pressed against the stop that is attached to the back side of the dildo and has a mechanism that allows to remove it or reattach to the dildo.

FIG. 4 shows the version of the invention with a limited axial movement of the additional element mounted on the dildo along the axis close to perpendicular of the longitudinal axis of the dildo.

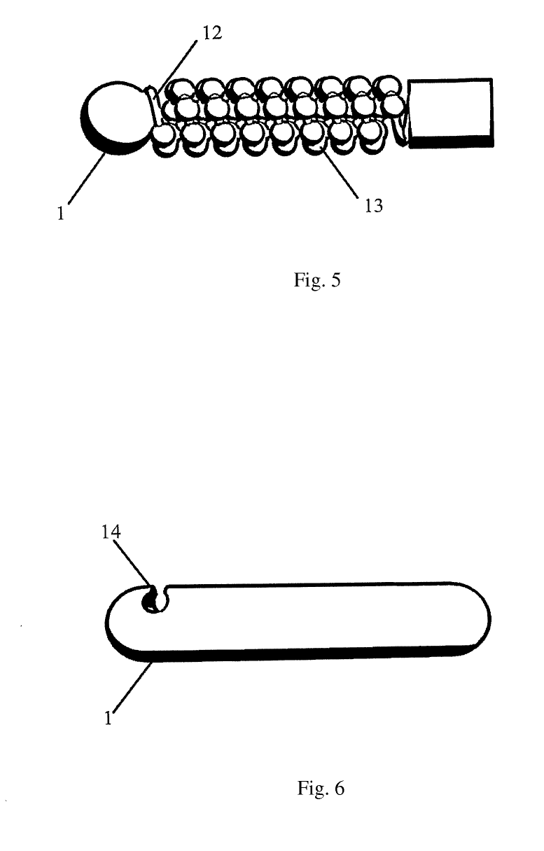

FIG. 5 shows the version of the invention with a few additional axial elements that are shaped like spheres with a central through hole and can be attached to the spiral-shaped body of the dildo.

FIG. 6 shows the version of the invention with the attachment part for an additional element.

Embodiment of the invention for the version shown in FIG. 1. The invention consists of a dildo (1) with a body of the additional element (2) with a clitoral stimulator (3) a spring-loaded guide (4) wavelike groove (5) pressing mechanism (6) and a stop (7).

Embodiment of the invention for the version shown in FIG. 2. The invention consists of a dildo (1) with a body of the additional element (2) with a clitoral stimulator (3) wavelike groove (4) pressing mechanism (5) a slot (6) and a ledge (7).

Embodiment of the invention for the version shown in FIG. 3. The invention consists of a dildo (1) with a body of the additional element (2) with a clitoral stimulator (3) wavelike groove (4) pressing mechanism (5) a stop (6) and a ledge (7).

Embodiment of the invention for the version shown in FIG. 4. The invention consists of a dildo (1) with an additional element (2) and an axis (3).

Embodiment of the invention for the version shown in FIG. 5. The invention consists of a dildo (1) with a spiral-shaped part (2) and additional spheres on it (3).

Embodiment of the invention for the version shown in FIG. 6. The invention consists of a dildo (1) with a locking mechanism (2).

Versions of the invention include a dildo with several undulating longitudinal grooves shaped like small wavelike canals with different lengths and/or heights of the waves and with a guide bearing. Notably, the body frame of the additional element is shaped like a ring and, on its side that faces the external genitalia, may have rollers or other rotary bodies that revolve along the axis close to the perpendicular of the longitudinal axis of the dildo, protrude above the additional element and are placed on two opposite sides relatively to the longitudinal axis of the dildo to thrust the body of the additional element against the vulva (labia majora) and allow it to move freely over it. The inner side of the dildo has a ledge that is spring-loaded or equipped with a mechanism that allows to move this ledge into or away from the body of the additional element enabling us to mound it in any recesses on the dildo, chose the angle of movement of the clitoral element or use it with no angle on a guide bearing.

Versions of the invention include a dildo with an additional element shaped like a ring that is worn on the dildo.

Versions of the invention include a single or double beam metallic spiral (i.e., a spiral made of metal wire with its both ends at one end of the spiral, but the other end of the spiral wire has a bending) with additional elements worn on it, that are shaped like spheres, disks, cylinders, ellipses and other rotary bodies, including hollow ones, and made of metal, plastic or glass.

Versions of the invention include a dildo with at least one locking mechanism and with at least one additional removable element designed for stimulation of erotogenic zones of external genitalia and internal genital organs, such as G-point. Notably, the additional stimulating element may have two stimulating surfaces that can be removed from or attached to the dildo by rotating them by 180 degrees along its longitudinal axis.

1-25. (canceled)

26. A device for stimulating erogenous zones comprising a dildo defined by an outer surface and longitudinal axis thereof, and at least one additional stimulating element,
the at least one additional stimulating element being attached to the dildo,
the at least one additional stimulating element including a body of rotation around an axis that is close to normal relative to the longitudinal axis of the dildo,
a surface of the at least one additional stimulating element projecting above the dildo outer surface,
whereby when in use the at least one additional stimulating element is able to roll over a surface of the erogenous zones.

27. The device according to claim 1, wherein the at least one additional stimulating element includes a ball.

28. The device according to claim 1, wherein the at least one additional stimulating element includes a cylinder.

29. The device according to claim 1, wherein the surface of the at least one additional stimulating element is made relief.

30. The device according to claim 1 wherein it is provided with a plurality of the additional stimulating elements and wherein at least a portion of the dildo includes a spiral frame, the spiral being fed through the plurality of the additional stimulating elements.
